(19) United States
(12) Patent Application Publication
(10) Pub. No.: US 2020/0261684 A1
(43) Pub. Date: Aug. 20, 2020

(54) SCENT POD AND A FACE MASK HAVING A SCENT POD

(71) Applicant: CANADA PROSPER APPAREL LTD., Kitchener (CA)

(72) Inventors: Steven Henry Fyke, Waterloo (CA); Jason Tyler Griffin, Kitchener (CA); Michael Joseph Defazio, Fonthill (CA); Richard Devin Szasz, Kitchener (CA); Peter Lionel Whitby, Kitchener (CA); Shannon Moore, Hamilton (CA); Martti Lemieux, Echo Bay (CA); Paul Kalbfleisch, Kitchener (CA)

(21) Appl. No.: 16/648,201

(22) PCT Filed: Sep. 18, 2018
(86) PCT No.: PCT/CA2018/051152

§ 371 Date: Mar. 17, 2020

Related U.S. Application Data
(60) Provisional application No. 62/559,709, filed on Sep. 18, 2017.

(51) Int. Cl. A61M 16/14; A61M 16/06; A62B 18/08; A62B 18/02

(52) U.S. Cl. CPC A61M 16/14; A61M 16/0683; A61M 2021/0016; A62B 18/025; A62B 18/08

(57) ABSTRACT

A scent pod is attached to a face mask. The scent pod includes a scented body for providing a scent to the user and a scent body holder for holding the scented body and securing the scented body to the face mask. A face mask includes a support for attaching to a user's face and a scent pod for providing a scent. The scent pod is attached to the support. The scent pod provides the scent to an internal chamber of the face mask.

### TECHNICAL FIELD

The embodiments disclosed herein relate to respirators and air monitoring systems, and, in particular to face masks for filtering air.

### INTRODUCTION

Respiratory masks are used in a wide variety of applications to protect a human's respiratory system from particles suspended in the air or from unpleasant or noxious gases. They are also frequently worn by, for example, medical care providers to prevent the spread of harmful micro-organisms either to or from the user.

Persons who work in polluted environments commonly wear filtering face masks to protect themselves from inhaling airborne contaminants. Filtering face masks typically have a fibrous or sorbent filter that is capable of removing particulate and/or gaseous contaminants from the air.

Respiratory masks have a variety of uses, including protecting a user from harmful bacteria or particles contained within unfiltered air. Existing respirators may not account for situations in which the filter degrades due to the pollutants filtered by the filter. These filtered pollutants cause the filter to degrade, requiring that the user receive filtered air through a degraded filter. This may require the replacement of the respiratory mask.

U.S. provisional patent No. 62/393,266, filed Sep. 12, 2016, entitled “face mask for filtering air and air monitoring system”, which is incorporated by reference in its entirety, describes a face mask.

### SUMMARY

According to some embodiments, there is provided a scent pod for attaching to a face mask. The scent pod includes a scented body for providing a scent to the user; and a scent body holder including: a body coupler for holding the scented body; and a mask coupler for securing the scent body holder to the face mask.

The scent may be any one or more of peppermint, eucalyptus, lavender, rosemary, chamomile, cinnamon, orange, menthol, cedar wood, bergamot, tangerine, ylang-ylang.

The body coupler may include a plurality of inwardly biased arms that hold the scented body.

The mask coupler may be spaced from the body coupler by a distance member. The mask coupler may include a connecting peg that engages with a connecting hole.

The scent body holder may include a scent diffusion aperture for enabling air to flow on multiple sides of the scented body.

The scented body may be shaped to correspond to an inner compartment of the body coupler.

The body coupler may include a scent pod clip for holding the scented body within the scent body holder. The scent pod clip may include a retention arm that transversely extends from the scent pod clip to pass around the scented body when the scented body is positioned within the body coupler.

The scented body may be secured around the body coupler. The scented body may be shaped to correspond to an outer surface of the body coupler.

The body coupler may be offset from a support such that there is space for a back surface of the scented body to slide around the body coupler.

The body coupler includes a scent pod clip for holding the scented body to the body coupler. The scented body has a pair of friction fit snaps that clip to scent pod clips that are located on opposite sides of the scented body.

According to some embodiments, there is provided a face mask including the scent pod.

According to some embodiments, there is provided a face mask including a support for attaching to a user's face and a scent pod for providing a scent, wherein the scent pod is attached to the support.

The scent pod provides the scent to an internal chamber of the face mask.

The scent pod may be positioned between the support and an air filter.

The scent pod may include a scented body for providing a scent to the user-facing chamber of the face mask.

The scent pod may be attached to an inner surface of the support.

The scent pod may be attached to a nose section of the support.

The scent pod may be attached at the intersection of a longitudinal support and a transverse support of the support.

The scent pod may be synchronized with a mobile device application. The mobile device application may recommend a scent based on an environmental property.

The scent pod may provide an increase in respiratory performance compared to respiratory performance without the scent pod.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatus of the present specification. In the drawings:

FIG. 1 is a perspective view of a face mask, according to one embodiment;

FIG. 2 is an exploded view of the face mask of FIG. 1;

FIG. 3 is a front view of a support for the face mask of FIG. 1;

FIG. 4 is a face-side view of the support of FIG. 3;

FIGS. 5A, 5B, 5C, and 5D are side, top, top perspective, and bottom perspective views, respectively, of a scent pod in an open position, in accordance with an embodiment;

FIGS. 6A, 6B, 6C, and 6D are side, top, top perspective, and bottom perspective views, respectively, of the scent pod of FIGS. 5A-5D in a secured position;

FIGS. 7A and 7B are perspective and detailed views, respectively, of a support and scent pod, in accordance with a further embodiment;

FIGS. 8A, 8B, and 8C are exploded, transparent, and perspective views, respectively, of a support and scent pod, in accordance with a further embodiment;

FIG. 9 is a face-side view of a face mask, in accordance with a further embodiment;

FIGS. 10A, 10B, 10C, and 10D, are mobile devices for a scent pod system, in accordance with an embodiment; and

FIGS. 11A and 11B illustrate front and face-side views, respectively, of a support having a scent pod, in accordance with an embodiment.

### DETAILED DESCRIPTION

Various apparatus or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

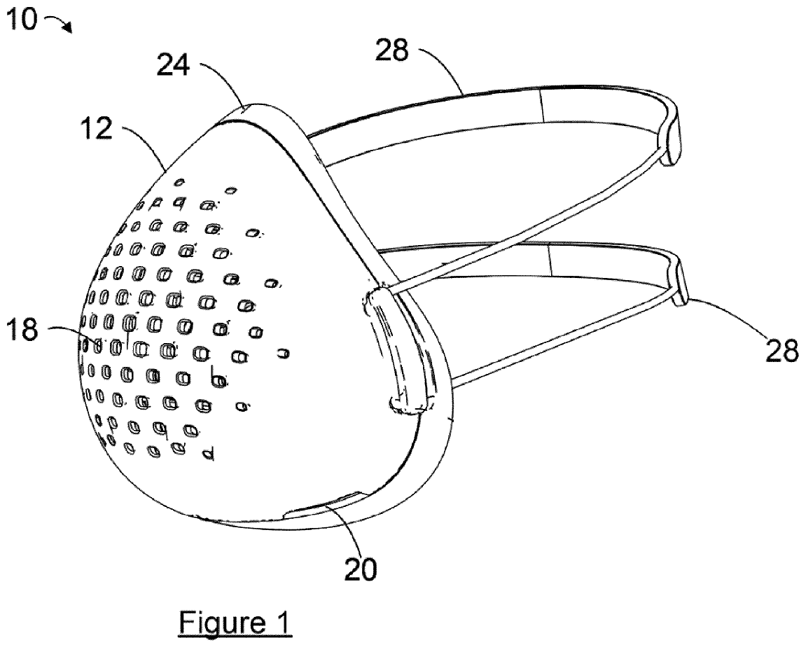

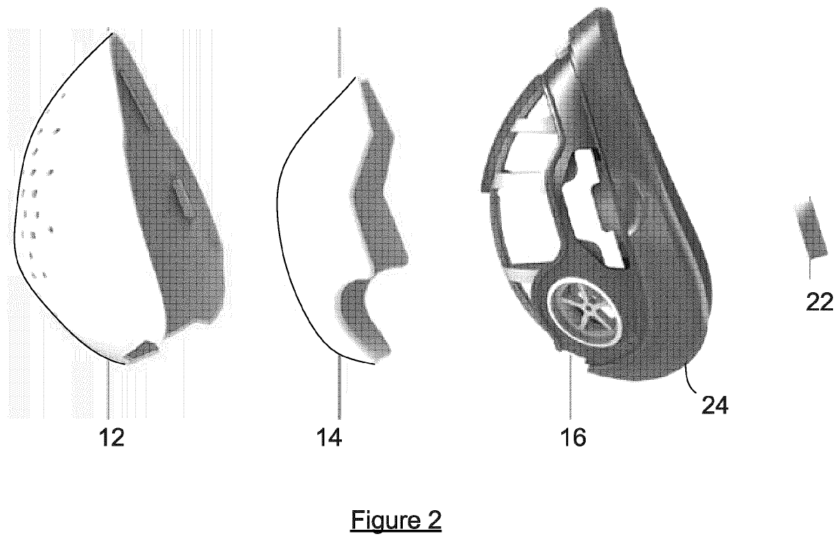

Referring to FIGS. 1 and 2, illustrated therein is a face mask 10 for filtering air. More particularly, the face mask 10 is a respiratory mask for filtering pollutants and particulate based airborne contaminants from the air when positioned over the face of a user. Air is drawn in by the user's breath and pollutants are filtered out and prevented from entering the respiratory system of the user. As the user exhales, the face mask 10 expels the exhaled air. The face mask 10 includes components that may provide ease of manufacture and simple assembly and replacement of parts for the user. Pollutants and airborne contaminants filtered may include carbon monoxide, nitrogen oxides, ozone, sulphur, PM2.5, PM10, and pollen. Pollen from plants may be filtered by the face mask 10 to prevent allergies and reactions in humans.

In an embodiment, the face mask 10 may filter any one or more of combustive atmospheric pollutants, motor vehicle emissions, power plant emissions, wood burning emissions, forest fire emissions, and agricultural burning emissions. The face mask 10 may be more effective than comparative products against <0.5 m airborne bacteria. The face mask 10 may be more effective then comparative products against <2.5 m combustive pollutants.

The face mask 10 includes a front shell 12 that acts as an outer layer of the face mask 10 and provides a protective outer surface. The front shell 12 attaches to a support 16 and the front shell 12 and the support 16 work together to hold a filter 14 therebetween. The support 16 provides structure to and supports the filter 14 in the face mask 10 while the front shell 12 protects the filter 14 and inner components of the face mask 10. The face mask 10 may include a head strap 28 attached to the front shell 12 for holding the face mask 10 to a user's head.

The front shell 12 has inlet holes 18 for allowing incoming air to pass in to the face mask 10 and through to the filter 14, where the filter 14 filters particulate elements from the air. The filter 14 is secured between the front shell 12 and the support 16.

The front shell 12 also has exhale ports 20 separate from the inlet holes 18, which allow exhaled air to pass out of face mask 10. The exhale ports 20 allow exhaled air to outlet the face mask 10 so that the exhaled air does not have to pass back through the inlet holes 18 thereby degrading the filter 14 from the inside.

The face mask 10 includes a face seal 24 attached to the periphery of the support 16 for providing a flexible and air-tight seal around the nose and mouth of the user. The face seal 24 provides a snug seal to the user's face and does not allow air to pass in through anything but the filter 14. The face seal 24 is located behind the front shell 12 and contacts the users face to make an air tight seal against the skin. This forces the intake air through the front shell 12 and through the filter 14. The seal 24 provides a high performance seal, creating a constrained and controlled environment.

The face mask 10 includes a scent pod 22. The scent pod 22 provides a scent. The scent may be an essential oil, a blend of various essential oils, or man-made created fragrances. The scent may be, for example, any one or more of peppermint, eucalyptus, lavender, rosemary, chamomile, cinnamon, orange, menthol, cedar wood, bergamot, tangerine, ylang-ylang or over 100 essential oils and various mix and blends of these oils. The scent could also be a man-made over the counter medicine or prescribed drug such as nicotine, ventolin or various antihistamines.

The scent may include scented liquid taken from certain plants using steam or pressure. Essential oils contain the natural chemicals that give the plant its “essence” (specific odor and flavor). Essential oils are used in perfumes, food flavorings, medicine, and aromatherapy.

The scent pod 22 may provide the delivery of over the counter drugs. The scent pod 22 may provide the delivery of prescription drugs (for example, Ventolin™, Nicotin, etc.).

In an embodiment, the scent is rosemary which has been scientifically proven to increase memory. In an embodiment, the scent is lavender which has been shown to reduce stress. In an embodiment, the scent is peppermint which has been shown to increase the lung capacity to breathe and intake oxygen. In an embodiment, the scent is Ventolin™ (albuterol) is a bronchodilator for relaxing muscles in the airways and increases air flow to the lungs. Accordingly, the scent pod 22 may provide therapeutic relief.

The scent may enhance performance. The scent pod may provide an increase in respiratory performance compared to respiratory performance without the scent pod. For example, the scent pod 22 may be used for everyday use, or for sporting or athletic applications such as jogging or running. While the scent pod 22 may provide a desirable scent, the scent pod 22 may also provide further functionally to increase, for example, athletic performance of a user. The face mask 10 and scent pods 22 may be used for adults or children.

The scent pod 22 is held in the face mask 10 in a constrained environment. The scent pod 22 may be attached to the face mask 10 in a number of ways including any one or more of clips or snaps, screws, fasteners, and magnets. This may prevent a user from choking on the scent pod 22.

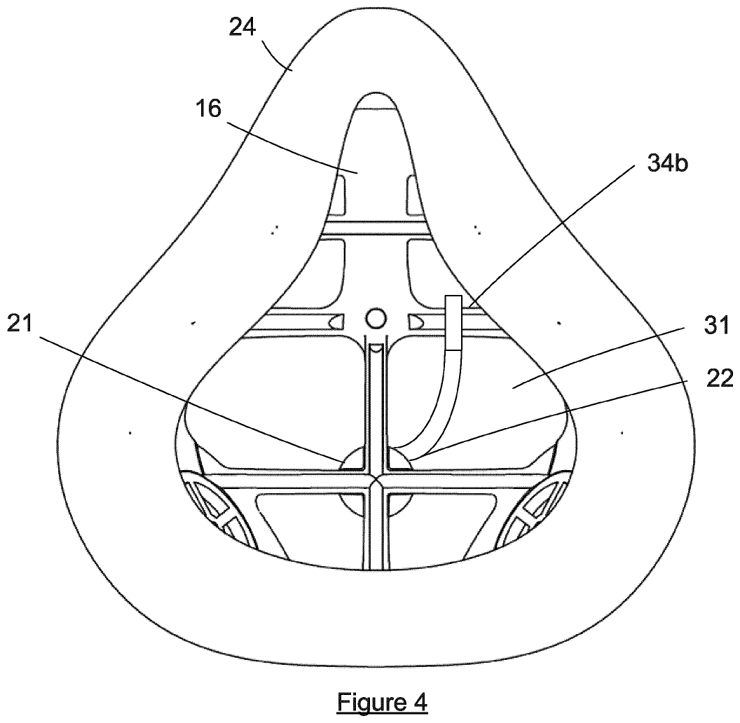

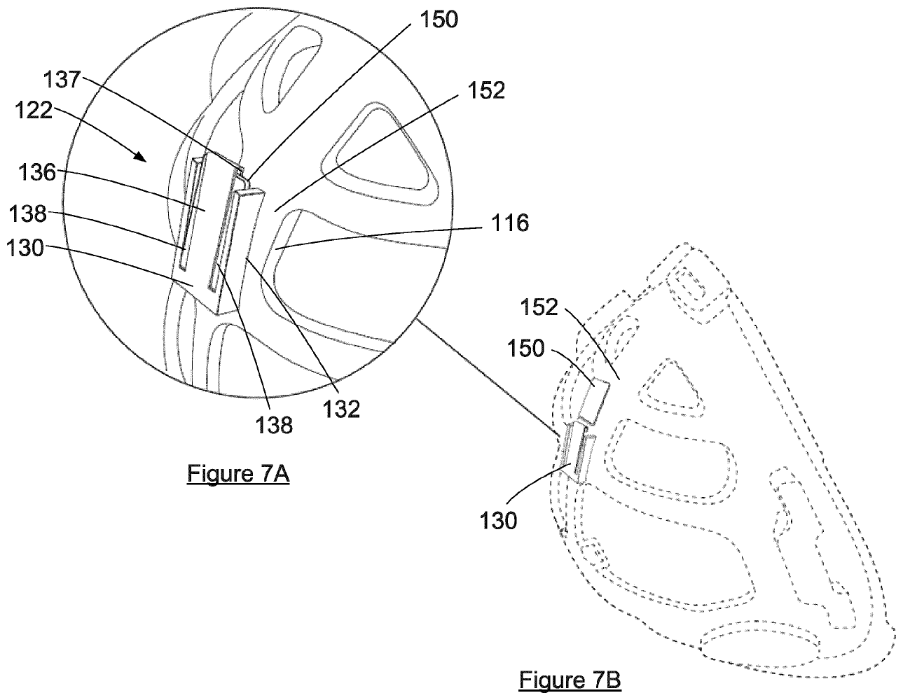

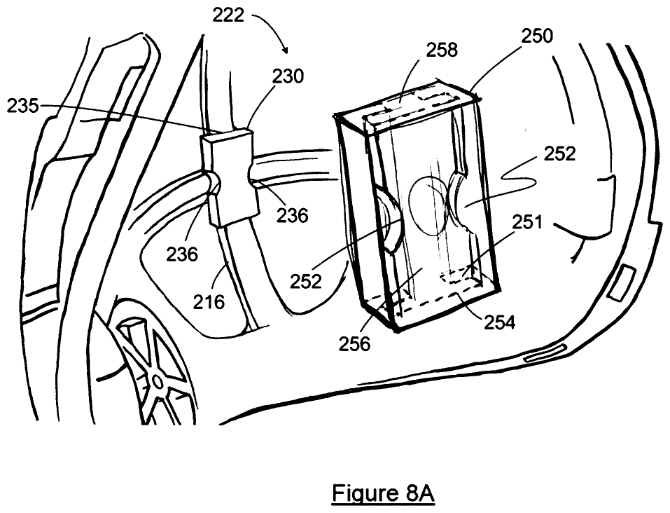

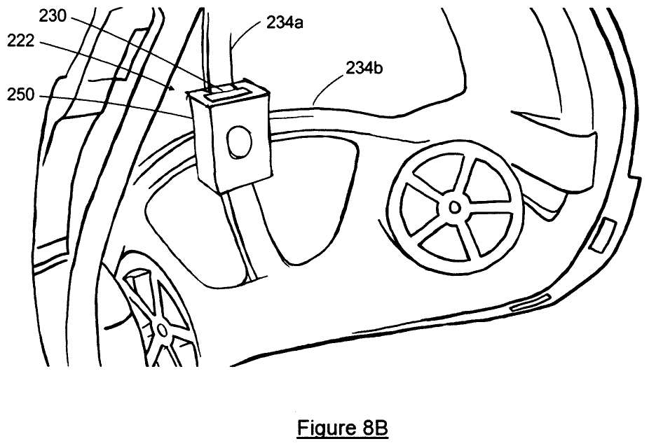

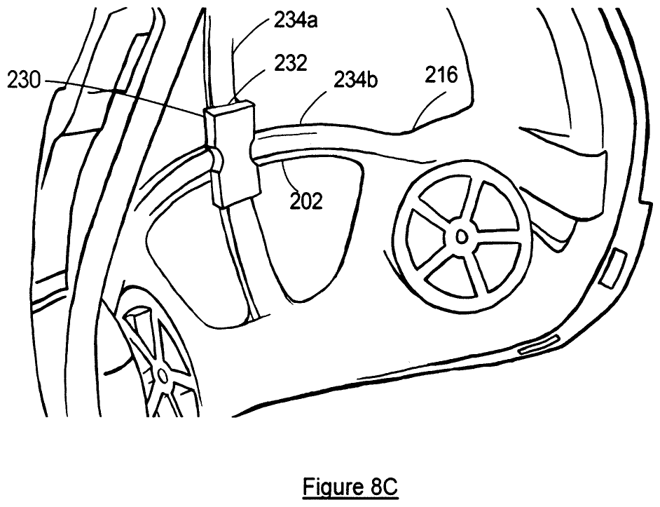

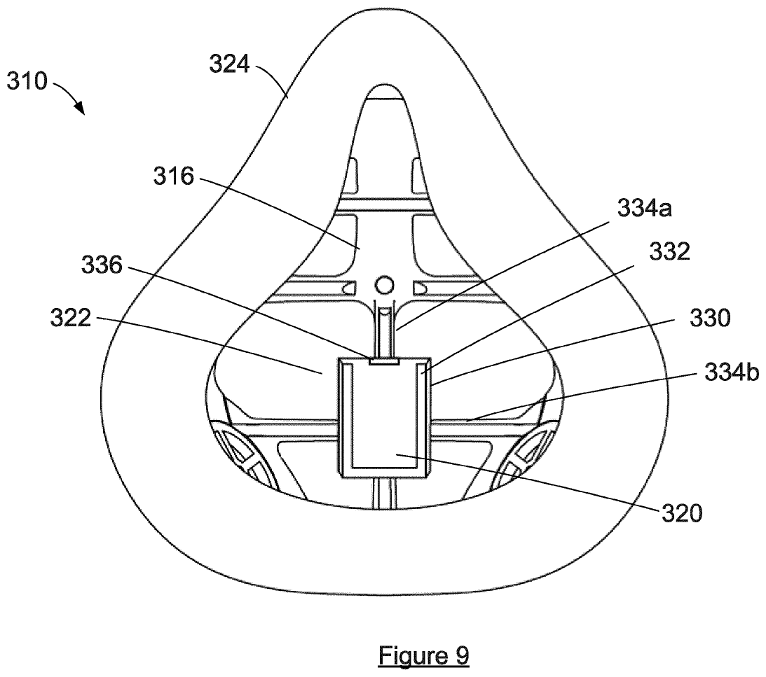

The scent pod 22 may be loosely attached to the support 16 and clipped to the lattice (FIGS. 4-6D). The scent pod 22 may be positioned between the support 16 and the filter 14 (FIGS. 7A and 7B). The scent pod 22 may be positioned between the support 16 and the user (FIGS. 8A-8C, FIG. 9).

The scent pod 22 provides a customizable environment within the face mask 10 for the user. In addition to filtering the air, the face mask 10 with the scent pod 22 provides a controlled environment for the user. As the airflow is controlled by the face seal 24, the airflow rate is controlled. As the airflow is controlled, the diffusion and delivery of the scent to the user may also be controlled.

In contrast to conventional masks, where the cloth surface abuts the user's face, the face mask 10 is spaced from the user's face and provides space for the scent pod 22 and airflow around the scent pod 22 to diffuse and suspend the scent in the air. This location may provide further enhanced performance for the user.

While the face mask 10 may filter air, even when used in non-polluted environments, the face mask 10 with scent pod 22 may provide an enhanced user experience.

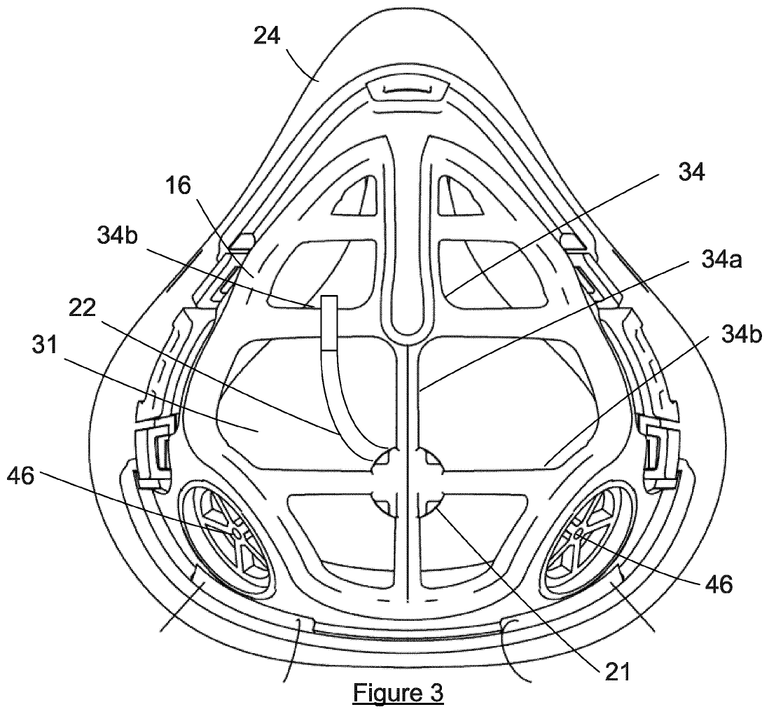

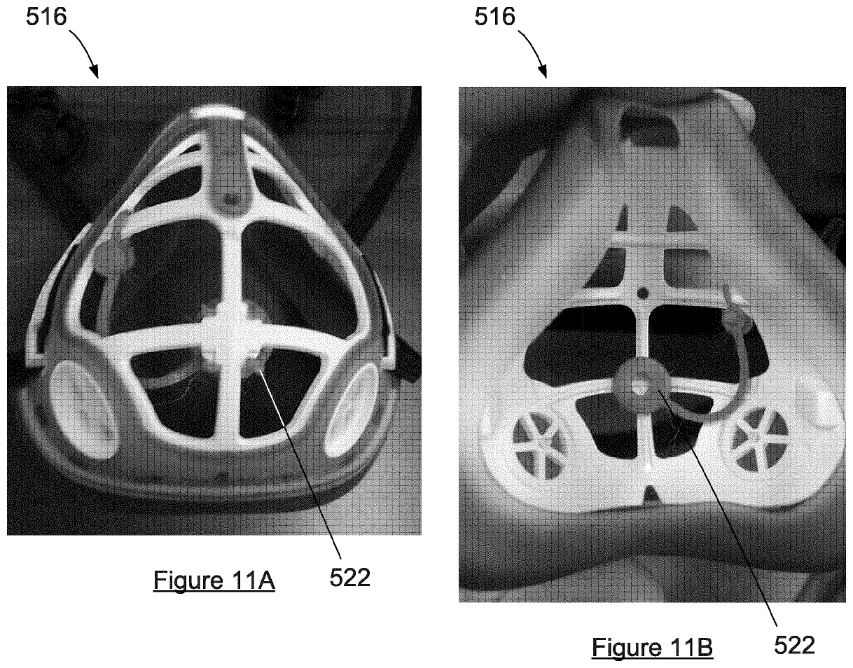

Turning now to FIGS. 3 and 4, illustrated therein is a face mask 10 including the support 16 and the face seal 24. FIGS. 11A and 11B illustrate an example of the support 516 (support 16) and the scent pod 522 (scent pod 22).

The support 16 includes a framework 34 having longitudinal 34a and transverse frame members 34b to support the filter 14 and open area 31 for allowing air to flow through. In an embodiment, the framework 34 includes a central longitudinal frame member 34a and at least two transverse frame members 34b. The filter 14 rests between the front shell 12 and the framework 34 and over the open area 31. The support 16 has exit valves 46 that lead to the exhale ports 20 on the front shell 12.

The scent pod 22 is coupled to the support 16. The scent pod 22 may be located along the inside surface of support 16. The scent pod 22 provides a scent to the internal chamber of the face mask 10 to give the user a more pleasant and comfortable experience.

The scent pod 22 includes a scented body 50 for providing a scent to the user-facing chamber of the face mask 10. The scented body 50 may be a diffusion pad. The scent pod 22 may include a scent body holder 26 for holding the scented body 50 and securing the scented body 50 to the facemask 10.

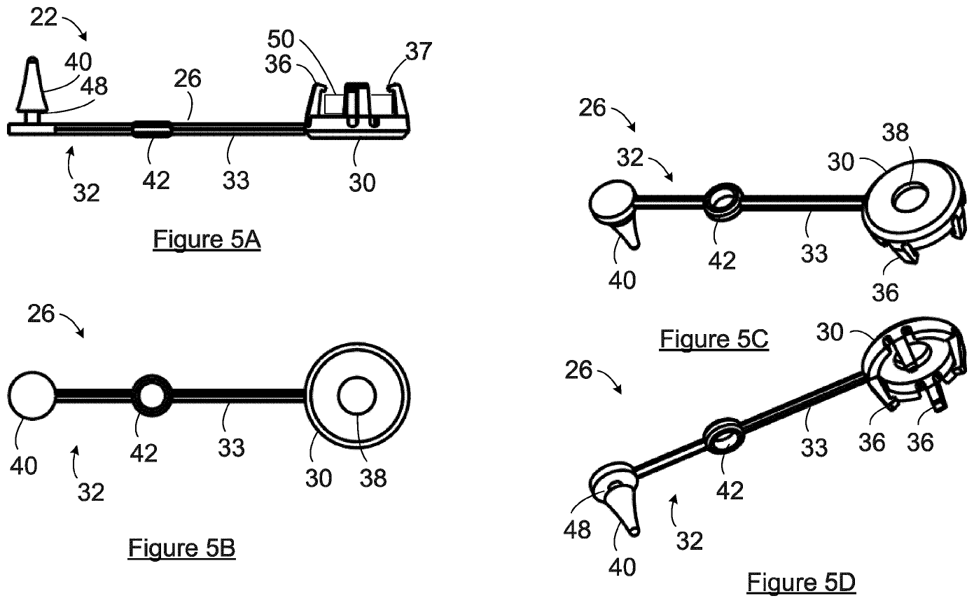

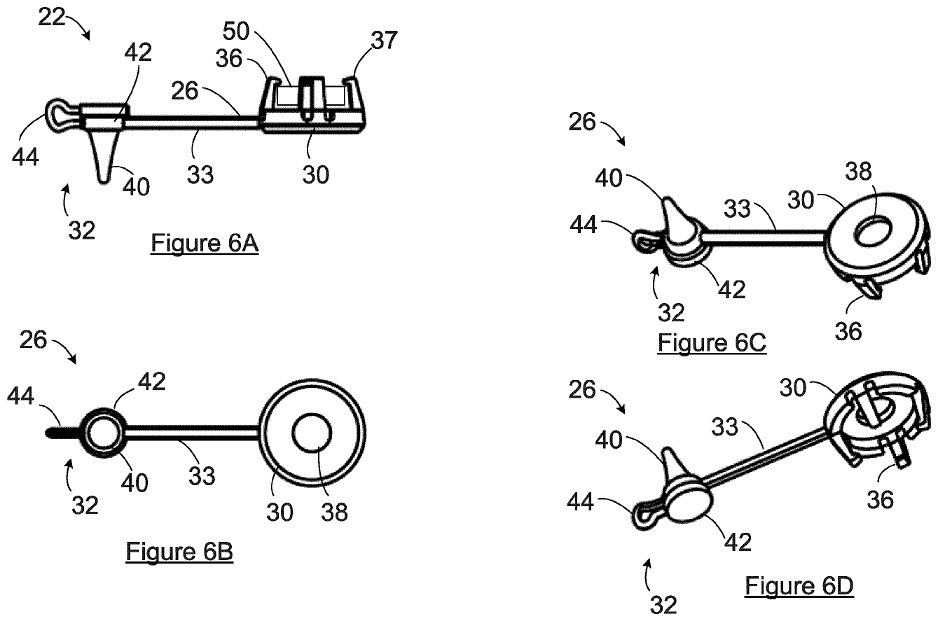

The face mask 10 includes a receiver 21 that connects with arms 36 (FIGS. 5A-6D) of the scent pod 22. The receiver 21 may be positioned at the intersection of the longitudinal 34a and transverse frame members 34b.

FIGS. 5A to 6D illustrate the scent body holder 26, in accordance with an embodiment. The scent body holder 26 secures a scented body 50 to the face mask 10. The scent body holder 26 includes a body coupler 30 located at a first end of the scent body holder 26 and a mask coupler 32 located at the other end of the scent body holder 26. The mask coupler 32 and the body coupler 30 may be spaced apart by a distance member 33 to distance the body coupler 30 from the mask coupler 32 and thus the face mask 10.

As shown at FIGS. 5A and 6A, the scent body holder 26 includes the body coupler 30 for securing the scented body 50. The body coupler 30 includes a plurality of inwardly biased arms 36 that hold the scented body 50. The arms 36 may be based to allow the insertion and extraction of the scented body 50 by a user. The scented body 50 is sized and shaped to fit into the scent body holder 26 and be secured to the scent body holder 26 while in use. The arms 36 may include perpendicular tips 37 that pass over and hold the scented body 50.

In addition, the arms 36 and tips 37 may also clip around the receiver 21 to secure the scented body to the support 16. The receiver 21 and body coupler 30, when clipped, are positioned at the intersection longitudinal 34a and transverse frame members 34b. In this way, the scent pod 22 is attached to the support 16 by two different mechanisms—the body coupler 30 to the receiver 21 and the mask coupler 32 to the framework 34.

The body coupler 30 may also include a scent diffusion aperture 38 for enabling air to flow on multiple sides of the scented body 50. The scent diffusion aperture 38 passes through the body coupler 30 and may also provide for the removal of the scented body 50, by a user.

The scented body 50 may be removed by a user by pulling back the arms 36, removing the body coupler 30 from the receiver 21, and optionally, pushing through the scent diffusion aperture 28, and sliding out the scented body 50. A new scented body 50 may be inserted.

The scent body holder 26 includes the mask coupler 32 for securing the scent body holder 26 to the support 16. In particular, the mask coupler 32 is configured to pass around the framework 34 of the support 16. In an embodiment, the mask coupler 32 is configured to pass around the longitudinal framework 34b. In an embodiment, the mask coupler 32 is configured to pass around the transverse framework 34a. The mask coupler 32 includes a connecting peg 40 that engages with a connecting hole 42.

FIGS. 5A to 5D illustrate the mask coupler 32 in an open and unsecured position while FIGS. 6A to 6D illustrate the mask coupler 32 in a folded and secured position. In the secured position, the mask coupler 32 is folded over and the connecting peg 40 passed through the connecting hole 42 and a loop 44 is formed which, when in use, loops around the support 16.

The connecting peg 40 may be made triangularly shaped and made of a flexible material. The connecting peg 40 includes a connecting recess 48 for securing with the connecting hole 42. The connecting peg 40 engages the connecting hole 42 by passing through the connecting hole 42, distal end first and once the connecting hole 42 has moved past the distal end, the connecting hole 42 is seated in a connecting recess 48. Once seated in the connecting recess 48, the connecting hole 42 secures the mask coupler 32 closed and in the secured position. The connecting peg 40 and connecting hole 42 are configured such that it is easier to close the mask coupler 32 than it is to open the mask coupler 32.

Turning now to FIGS. 7A and 7B, illustrated therein is a support 116 having a scent pod 122, in accordance with a further embodiment. The scent pod 122 includes a body coupler 130 and a scented body 150. The body coupler 130 is attached to an outer surface 152 of the support 116 of a face mask (such as the face mask 10 of FIG. 1). The scent pod 122 secures the scented body 150 to the face mask. The scented body 150 is shaped to correspond to an inner compartment of the body coupler 130. The scented body 150 may be a scent pad.

The scent pod 122 includes a mask coupler 132 for securing to the support 116. The mask coupler 132 may be integrally formed with the support 116. The mask coupler 132 may be affixed to the support 116 by adhesive or other bonding mechanisms.

The scent pod 122 may be attached to a nose section 152 of the support 116. This location of the scent pod 122 may provide increased scent directed towards the user's nose, where the scent will be most impactful. In addition, the nose section 152 may have an increased surface area on the support 116 that provides for increased space for location of the scent pod 122. The scent pod 122 is installed on the outside of lattice structure of the support, which is more accessible when the filter is removed.

The body coupler 130 includes a scent pod clip 136 for holding the scented body 150 within the scent body coupler 130. The scent pod clip 136 includes a retention arm 137 that transversely extends from the scent pod clip 136 to pass around the scented body 150, when the scented body 150 is positioned within the body coupler 130.

The scented body 150 may be removed by a user by pulling back the scent pod clip 136, pushing down on the scented body 150, and sliding out the scented body 150. A new scented body 150 may be inserted.

The body coupler 130 includes scent diffusion slots 138 to expose the surface of the scented body 150. The scent diffusion slots 138 provide for increased exposure of the scented body 150 that allows for airflow around the scented body 150 and through the body coupler 130.

Turning now to FIGS. 8A to 8C, illustrated therein is a support 216 having scent pod 222, in accordance with a further embodiment. The scent pod 222 includes a scented body 250 and a body coupler 230. The body coupler 230 is attached to an inner surface 202 of the support 216 of a face mask (such as the face mask 10 of FIG. 1). The body coupler 230 secures the scented body 250 to the face mask. The scented body 250 is secured around the body coupler 230. The scented body 250 is shaped to correspond to an outer surface of the body coupler 230.

The body coupler 230 includes a mask coupler 232 for securing to the support 216. The mask coupler 232 may be integrally formed with the support 216. The mask coupler 232 may be affixed to the support 216 by adhesive or other bonding mechanisms.

The body coupler 230 may be attached at the intersection of a longitudinal support 234a and a transverse support 234b. This location of the scent body holder 320 may provide increased scent directed towards the user's nose, where the scent will be most impactful. In addition, intersection of the longitudinal support 234a and the transverse support 234b may have an increased surface area on the support 216 that provides for increased space for location of the body coupler 230.

As shown at FIG. 8A, the body coupler 230 is offset from the support 216 such that there is space for the back surface 251 of the scented body 250 to slide around the body coupler 230. The body coupler 230 includes a scent pod clip 236 for holding the scented body 250 to the body coupler 230. The scent pod clip 236 mates with a friction fit snap 252 on the scented body 250. The scented body 250 has a pair of friction fit snaps 252 that clip to scent pod clips 236 that are located on opposite sides of the scented body 250.

The scented body 250 includes an open bottom aperture 254 that provides a slot opening to an interior cavity 256 of the scented body 250. The interior cavity 256 of the scented body 250 corresponds to the shape of the exterior surface of the body coupler 230. The scented body 250 includes a closed top surface 258 that rests on a top surface 235 of the body coupler 230.

The scented body 250 may be slid over the body coupler 230 and the friction fit snaps 252 of the scented body 250 snap to fit with the body coupler 230.

The scented body 250 may be removed by a user by pulling out the friction fit snaps 252 and sliding up the scented body 250 from the body coupler 230. A new scented body 250 may be inserted.

The scented body 250 may have an increased exposed surface area to proliferate the scent. Having the scented body 250 surround the body coupler 230 may provide for increased exposure of the scented body 250 that allows for airflow around the scented body 250 and increased scent diffusion.

FIG. 9 is a face mask support 310, in accordance with a further embodiment. The face mask support 310 is similar to the face mask support as described with reference to FIG. 4, except that the face mask support 310 includes a scent pod 322, in accordance with a further embodiment. The scent pod 322 includes a body coupler 330 and a scented body (not shown) such as a scent pad. The body coupler 330 is attached to an inner surface of the support 316 of a face mask 310 at the intersection of a longitudinal support 334a and a transverse support 334b. The body coupler 330 includes outer ridges 332 to hold the scent pad and a top flange 336 to secure the scent pad in place.

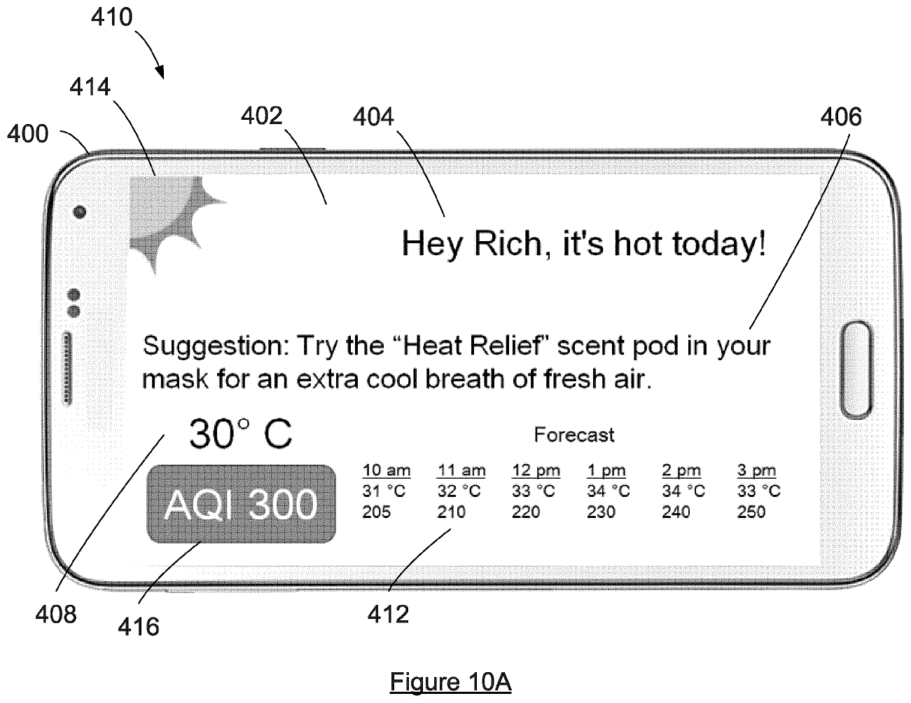

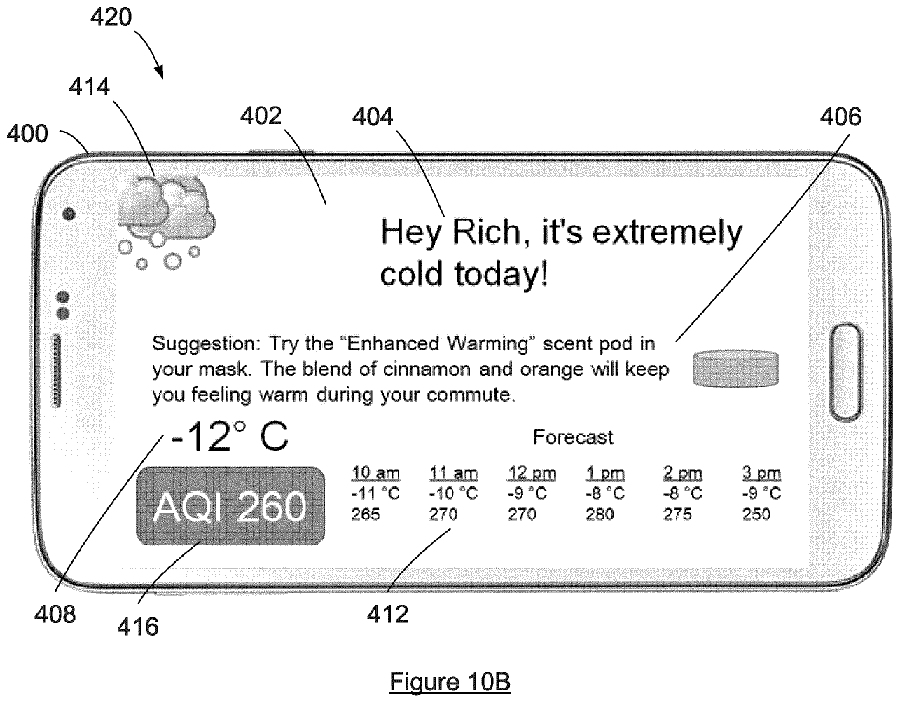

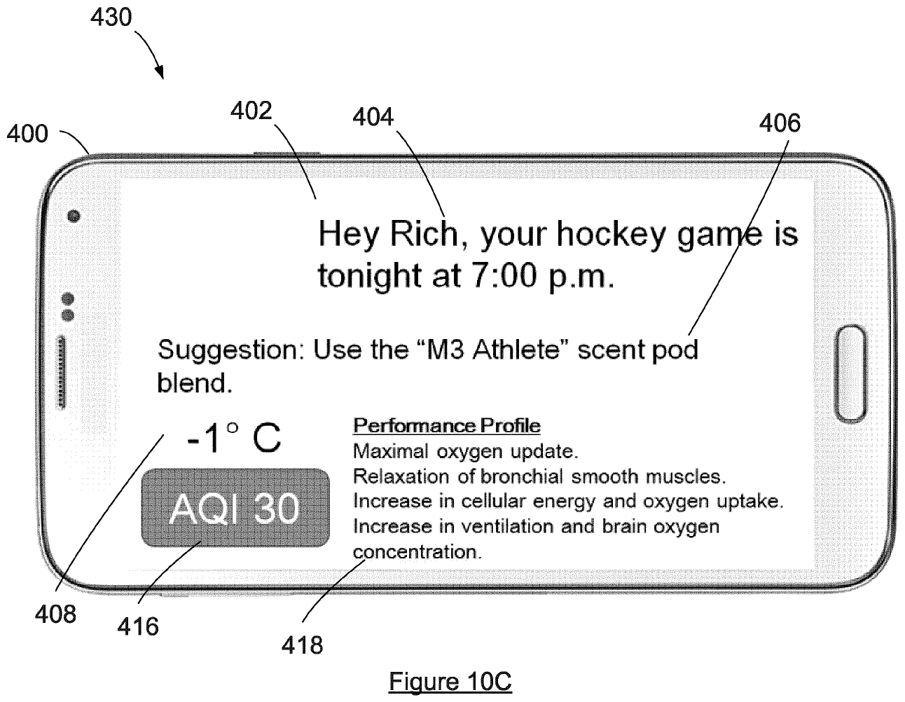

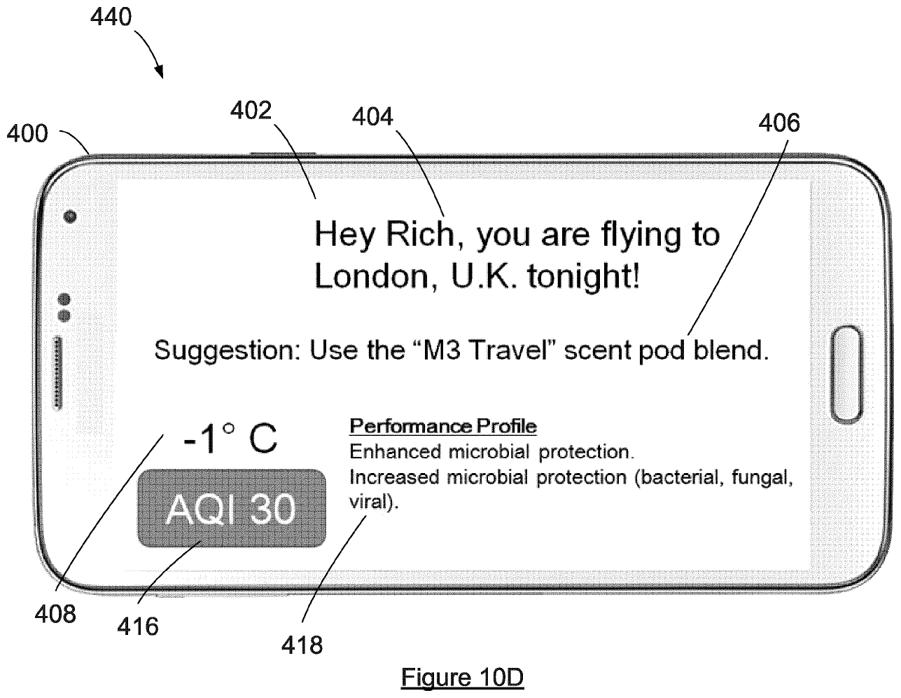

Turning now to FIGS. 10A-10D, illustrated therein is a user communication device 400, in accordance with an embodiment. The user communication device 400 may be part of an air enhancement system including the user communication device and a scent pod (such as those described above).

The user communication device 400 (such as a mobile device, personal computer, smartphone, or the like) my have Air Quality Reading data (AQRs). The user communication device 400 may be used by a user wearing or using the face mask. The user communication device 400 may communicate with other devices via a network.

The user communication device 400 includes a scent pod application 402 that is purpose built for interaction with the user. The scent pod is synchronized with the scent pod application 402. The scent pod application 402 may display, on the user communication device 400, various displays 410, 420, 430, 440. The scent pod application 402 may partner with the face mask in air pollution protection, and increase productivity and encourage maximal oxygen uptake or medicine doses.

The scent pod application 402 recommends a scent for the user to insert into the scent pod based on an environmental condition.

The display 410, 420, 430, 440 includes a user notification 404, and a scent pod suggestion 406. The user notification 404 may be based on the temperature 408, and the suggestion is based on any one or more of the temperature 408, the air quality index 416, and the forecast 412. The scent pod application 402 may also include an indicator 414 that graphically displays the weather conditions.

For example, the scent pod application 402 may recommend the “clear the air” scent when there is bad air pollution. The scent pod application 402 may recommend the “after the rain” blend when it is a rainy day. For example, the scent pod application 402 may display push notifications suggesting oils blends based on surrounding conditions such as weather, humidity, cold, and allergies.

For example, the display includes a user notification 404, and suggestion 406: Rich, it's going to be over 30 degrees this afternoon don't forget to bring the “heat relief” scent pod for the walk home with a blend of peppermint, mint and lemon you will feel cool and refreshed.

For example, the display includes a user notification 404: Hey Rich, It's cold today! and a suggestion 406: try using the “Warm drink” pod with Orange, lavender, coriander to help you stay warm during your morning commute.

For example, the display includes a user notification 404: Good afternoon Rich! and a suggestion 406: Help power through your afternoon by popping in the “energy and focus” blend to blast your productivity into the stratosphere!

For example, the display includes a user notification 404: Having trouble focusing right now at work? and a suggestion 406: Try out our guided breathing exercise combined “energy and focus” blend to take a 5 minute break and be mindful.

For example, the display includes a user notification 404: Hi Rich, you mentioned you have an allergy to pollen. The pollen report is going to be very bad for the next two days, and a suggestion 406: Make sure to wear your mask and try our “clear the air” scent pod blend.

The display 430, 440 may also include a performance profile 418 that describes the purpose and performance of the recommended scent pod.

The user communication device 400 may learn the user's patterns on when the user is using the scent pods and the user's specific health issues when it comes to breathing free and the scent pod application 402 may display a suggestion to use an oil that can help address these issues (for example clear up things like phlegm, or a sore throat).

The scent pod application 402 may include a feedback module that displays a question that asks if the particular suggested oil helped or not. Based on this response the scent pod application 402 learns and adapt for new suggestions in the future.

The interaction between the scent pod application 402 and the scent pod dynamically evolves as new scents are provided which can help people with different activities such as sports training, meditation, study, work, stress, sleep issues, respiratory or breathing issues, weather, allergies, immunity issues.

While the above description provides examples of one or more apparatus, methods, or systems, it will be appreciated that other apparatus, methods, or systems may be within the scope of the claims as interpreted by one of skill in the art.

1. A scent pod for attaching to a face mask, the scent pod including:
a scented body for providing a scent to the user; and
a scent body holder including: a body coupler for holding the scented body; and a mask coupler for securing the scent body holder to the face mask.

2. The scent pod of claim 1, wherein the scent is any one or more of peppermint, eucalyptus, lavender, rosemary, chamomile, cinnamon, orange, menthol, cedar wood, bergamot, tangerine, ylang-ylang.

3. The scent pod of claim 1, wherein the body coupler includes a plurality of inwardly biased arms that hold the scented body.

4. The scent pod of claim 1, wherein the mask coupler is spaced from the body coupler by a distance member, and wherein the mask coupler includes a connecting peg that engages with a connecting hole.

5. The scent pod of claim 1, wherein the scent body holder includes a scent diffusion aperture for enabling air to flow on multiple sides of the scented body.

6. The scent pod of claim 1, wherein the scented body is shaped to correspond to an inner compartment of the body coupler. The scent pod of claim 1, wherein the body coupler includes a scent pod clip for holding the scented body within the scent body holder, and wherein the scent pod clip includes a retention arm that transversely extends from the scent pod clip to pass around the scented body when the scented body is positioned within the body coupler.

8. The scent pod of claim 1, wherein the scented body is secured around the body coupler, and wherein the scented body is shaped to correspond to an outer surface of the body coupler.

9. The scent pod of claim 1, wherein the body coupler is offset from a support such that there is space for a back surface of the scented body to slide around the body coupler.

10. The scent pod of claim 1, wherein the body coupler includes a scent pod clip for holding the scented body to the body coupler, and wherein the scented body has a pair of friction fit snaps that clip to scent pod clips that are located on opposite sides of the scented body.

11. A face mask including the scent pod of claim 1.

12. A face mask including:
a support for attaching to a user's face; and
a scent pod for providing a scent, wherein the scent pod is attached to the support.

13. The face mask of claim 1 wherein, the scent pod provides the scent to an internal chamber of the face mask.

14. The face mask of claim 1 wherein, the scent pod is positioned between the support and an air filter.

15. The face mask of claim 1 wherein, the scent pod includes a scented body for providing a scent to the user-facing chamber of the face mask.

16. The face mask of claim 1 wherein, the scent pod is attached to an inner surface of the support.

17. The face mask of claim 1 wherein, the scent pod is attached to a nose section of the support.

18. The face mask of claim 1 wherein, the scent pod is attached at the intersection of a longitudinal support and a transverse support of the support.

19. The face mask of claim 1 wherein, the scent pod is synchronized with a mobile device application.

20. The face mask of claim 19 wherein, the mobile device application recommends a scent based on an environmental property.

21. The scent pod of claim 1 wherein the scent pod causes an increase in respiratory performance compared to respiratory performance without the scent pod.
